(19) United States
(12) Patent Application Publication
(10) Pub. No.: US 2024/0374910 A1
(43) Pub. Date: Nov. 14, 2024

(54) DEPRESSION TMS INDIVIDUALIZED TARGET LOCALIZATION METHOD AND SYSTEM BASED ON GROUP-LEVEL DIFFERENCE STATISTICAL MAPS

(71) Applicant: INSTITUTE OF PSYCHOLOGY CHINESE ACADEMY OF SCIENCES, Beijing (CN)

(72) Inventors: Chao-Gan YAN, Beijing (CN); Bin LU, Beijing (CN)

(73) Assignee: INSTITUTE OF PSYCHOLOGY CHINESE ACADEMY OF SCIENCES, Beijing (CN)

(21) Appl. No.: 18/660,395

(22) Filed: May 10, 2024

(30) Foreign Application Priority Data
May 10, 2023 (CN) 202310525347.7

(51) Int. Cl. A61N 2/00; G16H 20/40

(52) U.S. Cl. CPC A61N 2/006; G16H 20/40

(57) ABSTRACT

A method and system for individualized target localization for transcranial magnetic stimulation (TMS) in treating depression. The system acquires resting-state functional MRI (R-fMRI) brain imaging data from subjects in both a major depressive disorder (MDD) group and a matching normal control group, followed by data preprocessing. Taking the spherical subgenual anterior cingulate cortex (sgACC) as the seed point, functional connectivity calculations are performed for each subject, and sgACC functional connectivity maps within the mask of the dorsolateral prefrontal cortex (DLPFC) region are extracted. A two-sample t-test is conducted on the sgACC functional connectivity maps of the two groups to identify clusters within the DLPFC mask that show significant differences between the two groups, which are used as group-level localization targets. By integrating the obtained group-level localization targets with preprocessed individual MRI brain imaging data, individualized TMS targets are derived using a dual regression algorithm.

### TECHNICAL FIELD

This invention falls within the technical field of TMS target localization. Specifically, it relates to a method and system for individualized TMS target localization for depression based on statistical maps of group-level differences.

### BACKGROUND TECHNOLOGY

Repetitive Transcranial Magnetic Stimulation (rTMS) targeting the left DLPFC is a common treatment approved by the United States Food and Drug Administration (FDA) for treatment-resistant MDD.

At present, calculating functional connectivity (FC) based on functional magnetic resonance imaging (fMRI) is a common method for the precise localization of the DLPFC in the treatment of TMS for patients with treatment-resistant MDD. This method involves setting the sgACC as the region of interest (ROI), calculating functional connectivity at the whole-brain level, and then identifying the point of maximum negative connectivity within the DLPFC mask. However, due to the low signal-to-noise ratio of functional MRI imaging, using single-subject imaging to calculate TMS targets based on sgACC functional connectivity may yield unstable results. Therefore, researchers often use group-level average functional connectivity to determine corresponding targets. Neuroscience research has found a significant negative functional connection between the sgACC and the DLPFC, which is the target area for TMS treatment of depression. The location within the DLPFC area where this negative functional connection is maximized typically represents the optimal site for effective TMS stimulation in depression treatment.

However, these methods have certain limitations. One such limitation is the absence of a sizable collection of brain imaging data from patients diagnosed with MDD. Researchers use MRI data from healthy individuals to calculate whole-brain functional connectivity, but there is a significant difference between the whole-brain functional connectivity of patients with depression and that of healthy individuals. Therefore, TMS treatments guided by group-level DLPFC targets located using healthy samples may not achieve the best therapeutic effect for patients with depression. Additionally, this method calculates the DLPFC target based on group-level averaged whole-brain functional connectivity, which does not achieve individualized localization of the target.

### INVENTION CONTENT

To address the technical challenges present in the current technology, the present invention provides a method and system for individualized TMS target localization for depression based on group-level difference statistical maps, using brain imaging big data from thousands of depression samples to perform subsequent target calculations. This approach takes into full account the functional variability of spontaneous brain activity in patients with depression, making it more reasonable than algorithms based on healthy samples and achieving precise individualized localization for TMS treatment of MDD patients. The specific scheme adopted is as follows:

On the one hand, the present invention provides a method for individualized TMS target localization for depression based on group-level difference statistical maps, which includes the following steps:
Step 1, collect resting-state functional magnetic resonance brain imaging data from groups diagnosed with MDD and a matching normal control group;
Step 2, preprocess the collected resting-state fMRI brain imaging data;
Step 3, on the preprocessed individual resting-state fMRI brain imaging data, using the spherical sgACC as a seed point, calculate the seed point-based functional connectivity for each subject, and extract the sgACC functional connectivity map within the DLPFC region mask;
Step 4, perform a two-sample t-test on the sgACC functional connectivity maps of the MDD group and the normal control group. Within the DLPFC mask, extract clusters with significant differences between subjects in the MDD group and the normal control group from the two-sample t-test statistical map, and use them as the group-level TMS target localization for the MDD group;
Step 5, combine the obtained group-level TMS target localization for the major depressive disorder group with the preprocessed individual MDD resting-state functional magnetic resonance brain imaging data, and use a dual regression algorithm to obtain individualized TMS targets.

Further, the resting-state functional magnetic resonance brain imaging data collected in Step 1 comes from multiple sites, and the empirical Bayesian Combat algorithm is used for data normalization.

Further, in Step 2, the preprocessing of the collected resting-state functional magnetic resonance brain imaging data includes the following specific methods:
Step 2.1, remove the initial time points of the collected resting-state functional magnetic resonance brain imaging data to ensure magnetic field uniformity and subject adaptation to scanning conditions;
Step 2.2, convert the resting-state functional magnetic resonance brain imaging data into BIDS format and use preprocessing tools based on volume space or cortical space to preprocess the converted magnetic resonance structural and functional imaging data;
Step 2.3, use linear regression methods to denoise the preprocessed resting-state functional magnetic resonance brain imaging data;
Step 2.4, use a band-pass temporal filter and spatial smoothing methods to filter and smooth the resting-state functional magnetic resonance brain imaging data.

Further, in Step 3, on the preprocessed individual resting-state functional magnetic resonance imaging, use a spherical ROI of sgACC based in volume space or a template ROI of sgACC based in cortical space to calculate the whole-brain functional connectivity based on the sgACC seed point; use Pearson correlation to calculate functional connectivity.

Further, in Step 4, when performing a two-sample t-test on the sgACC functional connectivity maps of the major depressive disorder group and the normal control group, use a threshold-free cluster enhancement and permutation testing method or other correcting methods to perform multiple comparison corrections on the sgACC functional connectivity maps of the two-sample t-test. Within the DLPFC mask, extract clusters with significant differences between the corrected major depressive disorder group and normal control group, and use them as the group-level DLPFC TMS target for MDD treatment.

Further, the specific method for obtaining individualized TMS targets using a dual regression algorithm based on the group-level targets obtained in Step 5 includes:
Step 5.1, use the time series X⁽ⁱ⁾ of the whole brain voxel level after preprocessing of a single subject with major depressive disorder to form a two-dimensional matrix as the dependent variable;
Step 5.2, use the group-level localized target S^{(g)} as the independent variable, estimate the regression coefficients of the independent variable using the least squares method, and use it as the individual-level time series matrix A⁽ⁱ⁾ corresponding to the group-level localized target S^{(g)}, the expression is as follows:

A⁽ⁱ⁾=X⁽ⁱ⁾S^{(g),T}(S^{(g)}S^{(g),T})⁻¹
Step 5.3, use the individual-level time series matrix A⁽ⁱ⁾ corresponding to the group-level localized target S^{(g)} as the independent variable, apply the least squares method to obtain the time series individual-level target S⁽ⁱ⁾ corresponding to the group-level localized target S^{(g}). The expression is as follows:

S⁽ⁱ⁾=(A^{(i),T}A⁽ⁱ⁾)⁻¹A^{(i),T}X⁽ⁱ⁾
Step 5.4, extract the maximum value from the time series individual-level target S⁽ⁱ⁾, and use it as the individualized TMS target.

The invention also provides a system for individualized target localization in MDD TMS treatment based on group-level difference statistical maps. The system includes a computer and modules for data collection, data preprocessing, functional connectivity computation, statistical difference target acquisition, and individualized target acquisition:

The data collection module collects resting-state fMRI data of subjects from both a MDD group and a matched normal control group across various sites.

The data preprocessing module preprocesses the collected resting-state fMRI data.

The functional connectivity computation module calculates functional connectivity using the sgACC as the seed region for each subject on the preprocessed individual resting-state fMRI data, resulting in sgACC functional connectivity maps within the DLPFC region mask.

The statistical difference target acquisition module performs a two-sample t-test on the sgACC functional connectivity maps of both the MDD group and the normal control group. Within the DLPFC mask, it extracts significant clusters of differences between the subjects of both groups from the t-test statistical maps. These clusters are subsequently utilized as group-level localization targets for TMS in the MDD group.

The individualized target acquisition module combines the obtained TMS MDD group-level localization targets with preprocessed individual resting-state functional MDD fMRI data. It uses a dual-regression algorithm to determine individualized TMS targets.

Furthermore, the system includes a data normalization module running on the computer, which standardizes the data collected from different sites.

The technical solutions of the invention have the following advantages:
A. The invention utilizes a large sample of magnetic resonance brain imaging data from MDD subjects to construct individualized and precise TMS targets. Compared to the TMS targets constructed based on the brain imaging data from healthy individuals by previous methods, the targets are more specific to depression.
B. The invention employs the dual-regression algorithm as the core algorithm for individualized target localization. This algorithm takes into account both the robust group-level disease characteristics based on a large sample of the disease group and the specific disease variations of individual subjects, offering both stability and specificity.

Additionally, when performing the two-sample t-test on the sgACC functional connectivity maps of both the MDD and normal control groups, it employs a threshold-free cluster enhancement and permutation test method to accurately correct for multiple comparisons in the sgACC functional connectivity maps (voxel-p=0.001, cluster-p=0.05). This enhances the reliability and decreases the false-positive rate of the group-level targets, thereby making the localization of TMS targets more accurate.

### ILLUSTRATION EXPLANATION

To more clearly illustrate the specific implementations of the present invention, a brief introduction to the drawings required for these implementations is provided below. It is evident that the following drawings are some embodiments of the present invention. For those skilled in the art, other drawings can also be obtained based on these figures without creative efforts.

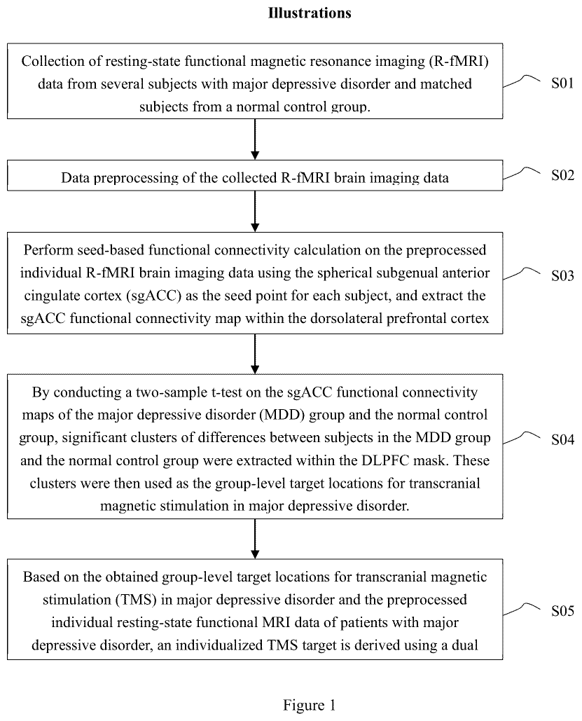

FIG. 1 is a schematic diagram of the depression TMS individualized target localization method based on group-level difference statistical maps provided by the present invention.

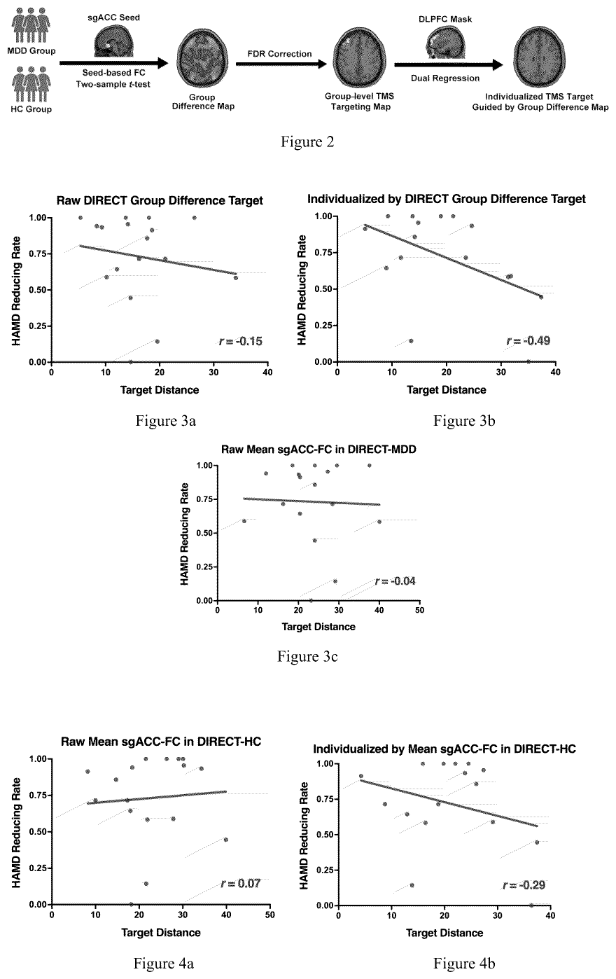

FIG. 2 is a process flowchart of the depression TMS individualized target localization method based on group-level difference statistical maps provided by the present invention.

FIG. 3a is an illustrative diagram of the group-level difference template localization effects provided by this invention.

FIG. 3b shows the individualized localization effect based on the difference template provided by the present invention.

FIG. 3c is an illustration of the localization effect based on the MDD (Major Depressive Disorder) average template.

FIG. 4a illustrates the localization effect based on the NC (Normal Control) average template.

FIG. 4b shows the individualized localization effect based on the NC individualized template.

FIGS. 5a and 5b respectively show the localization effects of the Weigand-2018 group-level target and Stanford Neuromodulation Therapy (SNT).

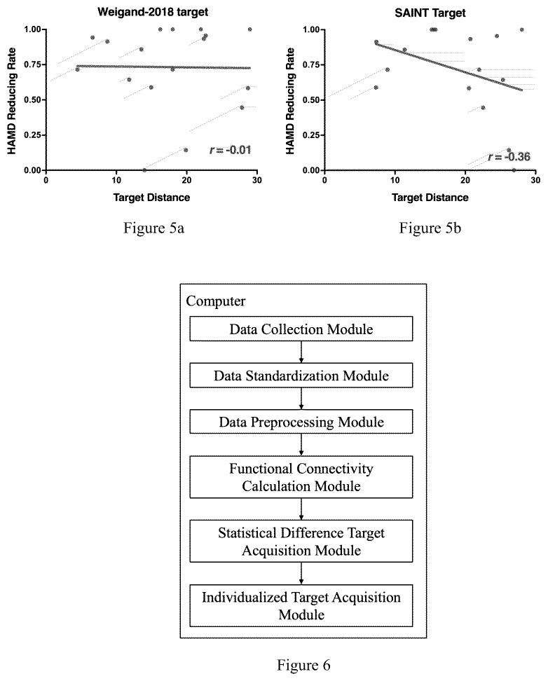

FIG. 6 is a structural diagram of the localization system composition provided by the present invention.

### DETAILED DESCRIPTION OF EMBODIMENTS

The technical solution of the present invention will be clearly and completely described in conjunction with the accompanying drawings. It is evident that the embodiments described herein are only part of the embodiments of the invention, and not all embodiments. All other embodiments obtained by those skilled in the art based on these embodiments of the invention, without any creative effort, fall within the scope of protection of the present invention.

As shown in FIG. 1 and FIG. 2, the present invention introduces an individual target localization method for TMS treatment of depression based on a statistical graph of group-level differences, which specifically includes the following steps:

[S01] Collect R-fMRI brain imaging data of subjects from an MDD group and a matching NC group.

Twenty-three research groups in China formed the depression imaging research consortium (DIRECT) and agreed to share resting-state fMRI indices from 1574 MDD patients and 1308 matched NCs. Since the data for the present invention come from multiple sites with different MRI scanners and scanning sequences, it is necessary to standardize data from different sites before subsequent processing. This method uses an empirical Bayesian-based Combat algorithm for data standardization. Age is included as a covariate in the Combat model, and the functional connectivity maps after Combat data standardization are included in subsequent calculations.

[S02] Perform data preprocessing on the collected resting-state functional magnetic resonance brain imaging data.

The present invention preferably uses DPABISurf for data preprocessing, which is a surface-based resting-state fMRI data analysis toolbox evolved from DPABI/DPARSF.

DPABISurf calls fMRIPrep to preprocess MRI structural and functional imaging data and provides a set of statistical and viewing tools.

Data preprocessing includes the following content:
(1) Remove the initial acquisition time points of the collected resting-state functional MRI brain imaging data, such as the first 10 time points, to ensure magnetic field uniformity and subject adaptation to scanning conditions;
(2) Convert the resting-state functional MRI brain imaging data into BIDS format, and use volume space or cortical space-based preprocessing tools to preprocess the converted MRI structural imaging and functional imaging data;
(3) Anatomical data preprocessing is as follows:
Perform intensity inhomogeneity correction on T1-weighted images with N4BiasFieldCorrection and use the corrected image as the T1w reference throughout the workflow;
Implement the antsBrainExtraction workflow with Nipype, using the OASIS30ANTs as the target template for skull stripping of the T1w reference;
Segment brain tissues into cerebrospinal fluid, white matter, and gray matter using FSL FAST;
Reconstruct the cerebral cortex with recon-all.
(4) Functional data preprocessing:
For each subject's resting-state functional MRI BOLD sequence, the following preprocessing is performed:
First, generate a reference volume and its skull-stripped version using the customized method of fMRIPrep;
Then, use bbregister (FreeSurfer) to coregister the BOLD reference to the T1w reference for boundary-based registration;
Apply time slice correction to the BOLD run with 3dTshift;
Resample the BOLD time series onto the fsaverage5 space surface.
(5) Noise regression:
Friston's 24-parameter model is used to regress head motion confounds. In addition, the mean frame-wise displacement is used to address the residual effects of motion in group analysis.

Other sources of noise signals (WM and CSF signals) are also removed from the data through linear regression to reduce the effects of respiration and cardiac activity. In addition, the linear trend was considered as a regressor to account for BOLD signal drift.
(6) Filtering and smoothing are as follows:
Finally, a band-pass time filter (0.01-0.1 Hz) and spatial smoothing (full width at half maximum, FWHM, 6 mm) are applied to functional images.
[S03] On the preprocessed individual resting-state functional MRI brain imaging data, using the sgACC as the seed region, perform seed-based functional connectivity calculations for each subject, and extract the sgACC functional connectivity maps within the DLPFC region mask.

### Functional Connectivity Calculation:

Using a volume-space based sgACC spherical ROI or a cortical space-based sgACC template ROI, calculate seed-based whole-brain functional connectivity on the large sample of MDD and NC preprocessed individual resting-state functional MRI brain imaging. The ROI coordinates are [6,16,−10], with a radius of 10 mm. Use Pearson correlation to calculate functional connectivity, and extract the functional connectivity patterns within the DLPFC region mask for subsequent calculations.

[S04] Perform a two-sample t-test on the sgACC functional connectivity maps of the MDD group and the NC group. Within the DLPFC mask, extract clusters that show significant differences between the MDD group and the NC group in the two-sample t-test statistical map, and use these clusters as the group-level target locations for TMS in the treatment of MDD.

Conduct a two-sample t-test on the sgACC functional connectivity maps for the MDD and NC groups, with gender, age, site, and scanning head motion as covariates in the general linear model to be accounted for; use threshold-free cluster enhancement and permutation tests to perform multiple comparison corrections on the two-sample t-test statistical map. Significant clusters are identified when both voxel-level p<0.001 and cluster-level p<0.05 are satisfied. Generally, the sgACC region is significantly negatively correlated with the potential TMS target regions in the DLPFC. Compared to healthy controls, the negative correlation between sgACC and DLPFC in MDD subjects is reduced. That is, there are regions in the sgACC functional connectivity map where the connectivity is significantly higher in the MDD group compared with the NC group. By stimulating these abnormal brain regions, the therapeutic effect for treating MDD is achieved. Within the DLPFC mask, extract clusters that show significant differences between the two groups after correction to serve as the group-level DLPFC TMS target for treating MDD. This group-level target is referred to as the “sgACC functional connectivity difference target between the MDD group and the NC group”, or simply “difference target”.

[S05] By integrating the obtained group-level target locations for TMS in MDD with the preprocessed individual resting-state functional MRI data of patients with MDD, an individualized TMS target is derived using a dual regression algorithm.

The TMS target locations for depression at the group level, based on large sample size parameter statistical maps, can fully capture potential targets related to the average abnormal state of spontaneous brain activity in depression. However, even within MDD group, there are differences in functional partitioning, localization, and abnormalities, necessitating individualization on top of the large-sample group-level target technology.

This invention employs dual regression for the individualization of targets, with the computational process for dual regression as follows:
The specific method for obtaining individualized TMS targets using the dual regression algorithm in Step 5 includes the following steps:
Step 5.1: Construct a two-dimensional matrix with the preprocessed whole-brain voxel-level time series X⁽ⁱ⁾ of an individual with MDD as the dependent variable.
Step 5.2: Use the group-level target locations S^{(g)} as the independent variable, employ the least squares method to estimate the regression coefficients, and take this as the individual-level time series matrix A⁽ⁱ⁾ corresponding to the group-level target locations S^{(g)}. The expression is as follows:

A⁽ⁱ⁾=X⁽ⁱ⁾S^{(g),T}(S^{(g)}S^{(g),T})⁻¹
Step 5.3: Use the individual-level time series matrix A⁽ⁱ⁾ corresponding to the group-level target locations S^{(g)} as the independent variable, and employ the least squares method to obtain the individual-level target locations S⁽ⁱ⁾ corresponding to the group-level target locations S^{(g)}. The expression is:

S⁽ⁱ⁾=(A^{(i),T}A⁽ⁱ⁾)⁻¹A^{(i),T}X⁽ⁱ⁾
Step 5.4: Extract the maximum value from the individual-level target locations S⁽ⁱ⁾ time series, and use this as the individualized TMS target.

To validate the effectiveness of the algorithm, this invention used a set of fMRI data from a group of 16 patients, all with Hamilton Depression Rating (HAMD) Scale scores greater than 7. TMS at a 5 Hz stimulation frequency was applied to the patients' DLPFC area, located using either the 5 mm rule or the EEG F3 site positioning. The patients' Hamilton Depression Rating Scale scores were recorded before treatment and after 6 months after treatment, and the reduction rate of these scores was used as an indicator of the therapeutic effect.

Using the subjects' resting-state functional magnetic resonance imaging obtained before treatment, the invention calculates the TMS MDD group-level target location based on a group-level statistical map derived from a large sample of depression patients. The Euclidean distance between the original stimulation target (based on traditional targeting methods) and the individualized precision target based on this invention was calculated. A smaller distance between these two points should correspond to a lower reduction rate in the HAMD scores (i.e., there should be a significant negative correlation between the TMS positioning offset distance and the reduction rate in HAMD scores). The greater the negative correlation between the TMS positioning offset distance and the reduction rate in HAMD scores, the better the targeting effect.

Test the positioning effect of the depression TMS individualized target location method based on the group-level difference statistical map proposed by this invention. To demonstrate that the individualized algorithm can enhance the effectiveness of group-level targets, the localization effects of unindividualized group-level targets based on large-scale statistical maps were also calculated. Furthermore, the localization effects of the targets calculated by this invention were compared with those of the internationally mainstream TMS DLPFC target for treating MDD (Weigand-2018 group-level target, MNI coordinates [−42 44 30]) and the targets used by the advanced Stanford Neuromodulation Therapy (SNT). The results showed:
The algorithm that achieved the best localization effect is the individualized localization method based on a differential template proposed by the present invention (r=−0.49), as shown in FIG. 3b. Moreover, the accuracy of the individualized localization method is higher than that of the corresponding group-level non-individualized version of the target. For comparison, FIG. 3a shows the group-level differential template localization effect (r=−0.15), and FIG. 3c shows the average template localization effect for MDD (r=−0.04).

As shown in FIGS. 4a and 4b, the positioning effects based on the NC (Normal Control) average template (r=0.07) and the NC individualized template (r=−0.29) were much lower than the individualized positioning effect based on the difference template proposed by the invention (r=−0.49). This proves the necessity of calculating the TMS target based on brain imaging data that includes a large sample of MDD, and that relying solely on the brain activity patterns of healthy subjects is insufficient to deduce the abnormal brain interaction patterns of MDD samples and calculate the optimal target.

As shown in FIGS. 5a and 5b, the positioning effect of the individualized localization method based on the difference template proposed by the invention was also far superior to the international leading Weigand-2018 group-level target (r=−0.01) and SNT therapy (r=−0.36), demonstrating the excellence of the individualized positioning method proposed by the invention.

As illustrated in FIG. 6, the invention also provides a depression TMS individualized target location system based on a group-level difference statistical map. The system includes a computer and data collection, data preprocessing, functional connectivity computation, statistical difference target acquisition, and individualized target acquisition modules running on the computer. The data collection module is used to gather resting-state fMRI brain imaging data of the major depressive disorder group and matched normal control group from various sites. The data preprocessing module processes the collected resting-state fMRI brain imaging data. The functional connectivity computation module calculates functional connectivity based on sgACC as the seed point for each subject on the preprocessed individual resting-state fMRI brain images, obtaining the sgACC functional connectivity map within the DLPFC region mask. The statistical difference target acquisition module performs a two-sample t-test on the sgACC functional connectivity maps of the major depressive disorder group and the normal control group. Within the DLPFC mask, it extracts clusters with significant differences between subjects in the major depressive disorder group and the normal control group from the two-sample t-test statistical map, which are used as group-level TMS targets for depression. The individualized target acquisition module combines the obtained depression group-level positioning targets and the preprocessed individual major depressive disorder magnetic resonance brain imaging individualized data, using a dual regression algorithm to obtain individualized TMS targets.

The system also includes a data standardization module running on the computer, which standardizes data collected from different sites for subsequent calculations.

This invention is based on large-sample MRI brain imaging data from patients with MDD and constructs individualized, precise positioning of TMS targets that possess greater specificity for depression compared to the TMS targets previously constructed based on MRI brain imaging data from healthy individuals. Aspects not mentioned in this invention are suitable for existing technology.

Clearly, the above examples are merely for the purpose of clear illustration and are not intended to limit the modes of implementation. For those skilled in the art, other different forms of changes or variations can be made based on the above description. It is neither necessary nor possible to exhaust all the implementation methods here. The obvious changes or variations derived from this are still within the scope of protection of this invention.

1-8. (canceled)

9. A method for individualized localizing the targets of transcranial magnetic stimulation (TMS) for depression based on group-level difference statistical maps, characterized by the following steps:
Step 1: Collect resting-state functional magnetic resonance imaging (R-fMRI) data from subjects diagnosed with major depressive disorder (MDD) and a matching normal control group;
Step 2: Preprocess the collected resting-state fMRI data;
The step 2 includes the following specific methods:
Step 2.1: Remove the initial time points of the collected resting-state fMRI data to ensure magnetic field homogeneity and subject adaptation to scanning conditions;
Step 2.2: Convert the resting-state fMRI data to BIDS format and use preprocessing tools based on anatomical or cortical space to preprocess the converted MR structural and functional imaging data;
Step 2.3: Employ a linear regression method to denoise the preprocessed resting-state fMRI data;
Step 2.4: Use a band-pass temporal filter and spatial smoothing methods to complete the filtering and smoothing of the resting-state fMRI data;
Step 3: On the preprocessed individual resting-state fMRI data, using the spherical subgenual anterior cingulate cortex (sgACC) as the seed point, calculate the seed-based functional connectivity for each subject, and extract the sgACC functional connectivity maps within the dorsolateral prefrontal cortex (DLPFC) region mask;
Step 4: Perform a two-sample t-test on the sgACC functional connectivity maps of the MDD group and the normal control group;
Within the DLPFC mask, extract significant clusters of differences between the subjects of the MDD group and the normal control group from the two-sample t-test statistical map and designate them as group-level targeting points for TMS in the MDD group;
Step 5: Combine the identified group-level TMS targeting points for MDD with the preprocessed individual MDD resting-state fMRI data and use a dual regression algorithm to obtain individualized TMS targets;
the method is characterized by the following steps to obtain individualized TMS targets through a dual regression algorithm based on the group-level localization targets obtained in step 5: Step 5.1, Use the preprocessed whole-brain voxel-level time series X⁽ⁱ⁾ of a single MDD subject to construct a two-dimensional matrix as the dependent variable;
Step 5.2, Use the group-level localized target S as the independent variable, estimate the regression coefficients of the independent variable using the least squares method, and use these coefficients as the individual-level time series matrix A corresponding to the group-level localized target S^{(g)}, expressed as follows:
A⁽ⁱ⁾=X⁽ⁱ⁾S^{(g),T}(S^{(g)}S^{(g),T})⁻¹
Step 5.3, Take the individual-level time series matrix A⁽ⁱ⁾ corresponding to the group-level localized target S^{(g)} as the independent variable, apply the least squares method to obtain the individual-level target S⁽ⁱ⁾ in the time series corresponding to the group-level localized target S^{(g)}, with the expression as follows:
S⁽ⁱ⁾=(A^{(i),T}A⁽ⁱ⁾)⁻¹A^{(i),T}X⁽ⁱ⁾∘
Step 5.4, Extract the maximum value from the individual-level target S⁽ⁱ⁾ and use it as the individualized TMS target.

10. The method according to claim 9, characterized in that the R-fMRI data collected in Step 1 come from multiple sites and are standardized using the empirical Bayesian Combat algorithm.

11. The method according to claim 9, characterized in that in Step 3, on the preprocessed individual resting-state fMRI data, use a spherical ROI of sgACC based on volume space or an sgACC template ROI based on cortical space to calculate the whole-brain functional connectivity based on the sgACC seed point; use Pearson correlation to calculate functional connectivity.

12. The method according to claim 9, characterized in that in Step 4, when performing the two-sample t-test on the sgACC functional connectivity maps of the MDD group and normal control group, use cluster enhancement and permutation testing method based on unthresholded cluster enhancement for multiple comparisons correction or gaussian random field correction of the sgACC functional connectivity maps from the two-sample t-test;
Within the DLPFC mask, extract the corrected significant clusters of differences between the MDD group and the normal control group as group-level DLPFC TMS targets for treating MDD.

13. A individualized TMS targeting system for depression based on group-level difference statistical maps, characterized in that the system includes a computer and data collection module, data preprocessing module, functional connectivity calculation module, statistical difference target acquisition module, and individualized target acquisition module running on the computer;
The data collection module is used to collect R-fMRI data of subjects from the MDD group and the matched normal control group across various sites;
The data preprocessing module is used to preprocess the collected rs-fMRI data;
The functional connectivity calculation module is used to perform functional connectivity calculations using the sgACC as a seed region on the preprocessed individual rs-fMRI data, to obtain a functional connectivity map of the sgACC within the DLPFC region mask;
The statistical difference target acquisition module is used to perform a two-sample t-test on the sgACC functional connectivity maps of the MDD group and the normal control group;
Within the DLPFC mask, significant differences clusters between subjects in the MDD group and the normal control group are extracted from the statistical maps generated by the two-sample t-test;
These clusters serve as group-level TMS targeting locations for the MDD group;
The individualized target acquisition module combines the obtained group-level TMS targeting locations for the MDD group and the preprocessed individual resting-state functional MDD magnetic resonance brain imaging data, using a dual regression algorithm to derive individualized TMS targets.

14. According to claim 13, the individualized depression TMS targeting system based on dual regression of two-sample groups, characterized in that the system further includes a data normalization module running on the computer, used for standardizing the data collected from different sites.
